(19) United States
(12) Patent Application Publication
(10) Pub. No.: US 2026/0174894 A1
(43) Pub. Date: Jun. 25, 2026

(54) EMBOLICS WITH IMMUNE-ENHANCING ANTI-CARCINOGENIC PROPERTIES

(71) Applicant: Bard Peripheral Vascular, Inc., Franklin Lakes, NJ (US)

(72) Inventor: Peter Maughan Crapo, Mesa, AZ (US)

(21) Appl. No.: 18/987,950

(22) Filed: Dec. 19, 2024

(51) Int. Cl. A61K 47/69; A61K 9/00; A61K 31/191; A61K 38/12; A61K 38/17; A61K 38/20; A61K 38/40; A61K 47/60; A61K 47/64; C07K 16/22; C07K 16/24; C07K 16/28

(52) U.S. Cl. CPC A61K 47/6903; A61K 9/0024; A61K 31/191; A61K 38/12; A61K 38/1709; A61K 38/2013; A61K 38/2046; A61K 38/208; A61K 38/40; A61K 47/60; A61K 47/643; C07K 16/22; C07K 16/247; C07K 16/248; C07K 16/2866

(57) ABSTRACT

Embodiments of the invention relate to embolic compositions having immunomodulating components therein and methods for using the embolic compositions to treat cancer. The embolic compositions include a hydrogel embolic with the immunomodulator(s) therein.

### BACKGROUND

The current standard of care for unresectable tumors is transarterial embolization (TAE) utilizing microbeads typically loaded with chemotherapeutic agents such as doxorubicin or radioisotopes such as Y90 depending on patient factors. Some emerging treatments are focused on embolization via hydrogels, shear-thinning materials, etc., that can dissipate in the blood to reduce collateral damage to nearby healthy tissues and may better resist reflux.

However, such techniques are often plagued by issues of over or under delivery of agents to the target tissue, such as a tumor.

### SUMMARY

Embodiments of the present disclosure relate to embolic compositions having immunomodulating components therein and methods of using the same. In an embodiment, an embolic composition is disclosed. The embolic composition includes a hydrogel embolic and at least one immunomodulator disposed within the hydrogel embolic. In embodiments, the at least one immunomodulator includes one or more of butyrate, hydroxybutyrate, at least one cecropin, at least one of a subset of interleukins, or one or more antibodies configured to block pro-cancer cell receptors.

In an embodiment, embolic microbeads are disclosed. The embolic microbeads include beads having at least one immunomodulator therein or thereon. In embodiments, the at least one immunomodulator includes one or more of butyrate, hydroxybutyrate, at least one cecropin, at least one of a subset of interleukins, or one or more antibodies configured to block pro-cancer cell receptors.

In an embodiment, a method of treating a tumor in a human body is disclosed. The method includes delivering an embolic composition having at least one immunomodulator thereon at least locally within the human body via an embolization. The embolic composition of the method includes a hydrogel embolic and at least one immunomodulator disposed within the hydrogel embolic. In embodiments, the at least one immunomodulator includes one or more of butyrate, hydroxybutyrate, at least one cecropin, at least one of a subset of interleukins, or one or more antibodies configured to block pro-cancer cell receptors. The method may further include preparing the embolic composition.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the invention, wherein identical reference numerals refer to identical or similar elements or features in different views or embodiments shown in the drawings.

FIG. 1 is a flow chart of a method for treating a tumor in a human body, according to an embodiment.

### DETAILED DESCRIPTION

Embodiments of the present disclosure relate to incorporation of immunoregulating (e.g., immune-enhancing) compositions within an embolic agent, such as hydrogel or microbead embolics having the immunoregulating compositions therein. The embolic agents may be in the form of one or more of hydrogel or microbeads composed to embolize tissue within the human body and delivery the immunoregulating composition to the tissue.

Arterial embolization is designed to fill target vessels to occlude flow, thereby limiting nutrient and oxygen supply to the target area (e.g., tumor). However, the mode of treatment—systemic delivery—and the agents delivered typically damage both cancerous and healthy tissue indiscriminately.

The immunomodulator(s) within the embolic agents disclosed herein recruit the body's anti-carcinogenic defenses to improve treatment efficacy and to reduce collateral damage in comparison to alternative agents. The embodiments disclosed herein provide for local delivery of an immune-enhancing agent (e.g., biomolecule), that is typically delivered systemically, to reduce side effects and increase efficacy. For example, a number of clinical trials for potential immunotherapies have been discontinued due to severe side effects during systemic administration. The local delivery enabled by the embodiments herein significantly reduce or altogether avoid side effects, increase efficacy, and reduce costs of therapy compared to other treatments without the local delivery of embolics having immune-enhancing agents therein.

As noted above, the embolic composition may include a hydrogel composition, a particle composition, or both, carrying at least one immune-enhancing agent or immunomodulator therein.

### Hydrogel

In some embodiments, the embolic composition includes a hydrogel embolic and at least one immunomodulator (e.g., immune-enhancing agent) disposed within the hydrogel embolic. The at least one immunomodulator may include one or more of butyrate, hydroxybutyrate, at least one cecropin, at least one of a subset of interleukins, one or more antibodies configured to block pro-cancer cell receptors, or any other immunomodulator(s). The at least one immunomodulator may include one or more small molecules having immunomodulatory or anti-cancer properties. The concentration of the at least one immunomodulator in the embolic composition may vary depending upon the specific immunomodulator(s) present. Various immunomodulators are disclosed in more detail below.

The hydrogel embolic includes a hydrogel composed to be used as, or deliver an embolic, to a selected region or a local tissue within the human body. The hydrogel embolic may include any commercial hydrogel embolic into which the at least one immunomodulator may be disposed (e.g., bonded, entangled in).

In some embodiments, the hydrogel embolic may include a hydrogel having at least one complexing component and at least one branching component bonded to the at least one complexing component to form a hydrogel backbone.

The complexing component may include one or more peptides, polypeptides, or proteins. A peptide includes to two or more amino acids linked via a peptide bond, a polypeptide includes a series or chain of multiple linked amino acids, and a protein refers to a full-length expressed gene or chimera thereof. In some examples, the complexing component may include a protein, such as a recombinant protein and/or synthetic protein. The protein may include a commonly found protein in humans. Such common protein(s) may be desirable because they produce limited response by the immune system of humans. In some examples, the complexing component includes an albumin, such as human albumin, human serum albumin (HSA), recombinant human serum albumin (rHSA), synthetic human serum albumin, or the like. In some examples, the complexing component may include poly-lysine or a polypeptide with multiple lysine residues. Such examples include amine groups as reactive side chains available to react with (e.g., cross-link to) the branching component (e.g., PEG-NHS ester of the branching component).

In some examples, the complexing component (e.g., complexing molecule) has a molecular weight (MW) of about 1,000 Da to about 100,000 Da, such as about 1,000 Da to about 20,000 Da, about 20,000 Da to about 50,000 Da, about 50,000 Da to about 70,000 Da, about 60,000 Da to about 80,000 Da, about 70,000 Da to about 100,000 Da, less than 100,000 Da, or less than 70,000 Da. For example, rHSA has a MW of about 66 kDa. PEI has a variable MW depending on the length of the monomer. Similarly, a poly-lysine polypeptide MW can be constructed based on a desired size or MW. Such a MWs may be similar or identical to the any of the MWs disclosed above with respect to albumin.

The complexing component may be provided in an amount effective to result in a (hydrated) hydrogel having about 2 to about 60 weight % (wt %) complexing component, such as about 2 wt % to about 10 wt %, about 3 wt % to about 8 wt %, about 5 wt % to about 15 wt %, about 15 wt % to about 25 wt %, about 10 wt % to about 20 wt %, about 15 wt % to about 35 wt %, about 20 wt % to about 40 wt %, about 30 wt % to about 50 wt %, about 40 wt % to about 60 wt %, less than 60 wt %, less than 40 wt %, or less than 10 wt % of the hydrogel.

The complexing component may be provided in dry form and may be hydrated before or contemporaneous with deployment of the hydrogel embolic into the human body. The complexing component maty be disposed in a basic solution, such as at a pH of at least 7.5, at least 8.5, at least 9.0, or at least 9.5.

The at least one branching component is formulated or otherwise configured to bond with (e.g., cross-link to) the at least one complexing component. Accordingly, when reacted together the at least one branching component and the at least one complexing component cross-link to form the hydrogel backbone.

The branching component may include a plurality of chemical species (e.g., monomers, oligomers, or polymers) composed to bond with the complexing component. As explained in more detail below the branching component may include a polyethylene glycol, such as a multi-arm PEG having one or more tethering groups thereon. The one or more tethering groups are composed to bond with (e.g., cross-link with) one or more functional groups of the complexing component.

The branching component(s) may include a linker (or a spacer) with moieties along the length thereof for binding other branching components, complexing components, or water. For example, the branching component(s) may include a PEG or repeating units thereof, attached to a core or central moiety at one end and a tethering group at the opposing end.

Formula I below is an example structure of the branching component:

(Core)-(Linker)ₙ-(Tethering Group) (I)

The number n may vary depending on the desired final molecular mass of the branching component or resulting hydrogel. For example, n may be an integer from 1 to 1,000, such as 1 to 100, 100 to 200, 200 to 300, 300 to 400, 400 to 500, 500 to 1,000, at least 20, at least 25, at least 40, at least 50, at least 75, at least 100, less than 1,000, less than 500, less than 200, less than 100, or less than 50.

The core may include a hydroxyl or polyol, such as pentaerythritol, hexaglycerol, tripentaerythritol, or glycerol. In some examples, the core is glycerol-based core allowing for multiple “arms” to extend therefrom. For example, a glycerol may provide for two arms, pentaerythritol can provide up to four arms, hexaglycerol can provide up to eight arms, and tripentaerythritol can provide up to eight arms.

The linker provides a connection between the tethering group and the core. The linker may include an ethylene glycol, a PEG, or a linear or branched chain thereof. For example, a PEG-chain may link the tethering group to the core. The linker may be a PEG sufficient to provide a molecular weight (MW) of at least about 1,000 Daltons (Da) to the PEG or the resulting branching component, such as about 1,000 Da to about 100,000 Da, about 1,000 Da to about 10,000 Da, about 10,000 Da to about 20,000 Da, about 15,000 Da to about 25,000 Da, about 20,000 Da to about 40,000 Da, about 40,000 Da to about 60,000 Da, about 60,000 Da to about 80,000 Da, about 80,000 Da to about 100,000 Da, about 1,000 Da to about 50,000 Da, about 50,000 Da to about 100,000 Da, less than about 100,000 Da, or less than about 50,000 Da. Selective control of the MW of the PEG in the linker can allow for the hydrogel to retain a selected amount of water, such as a greater MW corresponding to a greater amount of water retained.

The linker may have one or more functional groups or moieties at the terminal ends thereof composed to bond with the tethering group and core. The linker may be composed to bond with a reactive moiety of the tethering group to form the branching component. Such reactive moieties may include an amine, an alkene, an alkyne, or the like. In some examples, the linker may include one or more carbonyl containing group(s), such as at least one carboxylic acid, ester, acid halide, aldehyde, or the like.

The tethering group of the branching component is composed to covalently bond with one or more moieties of the complexing component upon mixing.

The tethering group may include an ester comprising the carbonyl group or ester of the linker. For example, the tethering group may include a succinimidyl ester of the PEG linker. In such examples, the succinimide may be N-hydroxysuccinimide (NHS). The succinimide may be a further substituted NHS. The tethering group may include a succinimidyl carboxymethyl (SCM), a succinimidyl succinate (SS), a succinimidyl glutarate (SG), or the like. In some examples, the tethering group includes at least one of NHS-SG, NHS-SS, and NHS-SCM. The tethering group may include a maleimide (e.g., maleimide bonded to the PEG linker), propionamide (e.g., propionamide bonded to the PEG linker).

In some examples, the tethering group may include a cyclical or ringed organic compound conjugated to the linker through a carboxyl group or an appended carboxyl group of a carboxylic acid. For example, a succinimide is bonded to the carboxylic acid via the nitrogen heteroatom in the five-member ring. In some examples, the tethering group may include BA, NHS, acrylamide, an α-haloacetyl, biotin, a carbodiimide, a carboxylic acid, an alkyne, a halogen (such as chloride), an epoxide, hydrazide, norborene, hydroxyl, azide, amine, acrylate, dibenzocyclooctyne (DBCO), glutamic acid, glutaramide acid, a succinimide (e.g., succinimidyl glutarate (SG), succinimidyl glutaramide (GAS)), a maleimide, para-nitrophenyl carbonate (NPC), a propionamide, a pyridyl disulfide (e.g., orthopyridyl disulfide (OPSS)), acetic acid, carboxyl methyl, glutaric acid, succinic acid, glutaramide acid, succinamide acid, succinimidyl succinamide (SAS), succinimidyl carboxymethyl (SCM) ester, thiol (or SH), tosylate, vinylsulfone, or combinations thereof. The tethering group may include esters or any of the foregoing.

In some examples, the tethering group is linked to an alkanedioic acid, such as a linear dicarboxylic acid. In some examples, the linear dicarboxylic acid is saturated. In some examples, the dicarboxylic acid is unsaturated. The alkanedioic/alkenedioic/branched acid may include one or more of oxalic acid, malonic acid, succinic acid, itaconic acid, glutaric acid, adipic acid, pimelic acid, suberic acid, azelaic acid, tartaric acid, malic acid, or citraconic acid. The primary amine or heteroatom of a ringed organic compound (e.g., succinimide) is linked to the dicarboxylic acid. In some examples, the tethering group is an ester of SG or NHS and one of glycolic acid, succinic acid, and glutaric acid.

The linker and/or tethering group may provide reactive moieties for cross-linking therebetween, such as exposed oxygens, nitrogens, sulfurs, hydroxyl groups, carbonyl carbons, amines, and the like. For example, the linker may include one or more ethylene glycol molecules or PEGS.

The branching component may include a multi-arm molecule, such as a multi-arm PEG. One or more linkers may bind to the multi-arm core and extend outward therefrom. Two, four, six, or eight linkers may be attached to the core. Each linker connected to the core may be considered as an appendage or arm extending therefrom. At least some of the linkers in the multi-arm PEG may include a tethering group bonded thereto.

While described above as having the configuration of a core-linker-tethering group, the branching components may have multi-armed configuration having a core linked to two or more linkers. For example, the branching component may have the core including 2 arms with the structure of:

(Tethering Group)-(Linker)-(Core)-(Linker)-(Tethering Group) (II).

In such a two-armed structure, a single ethylene glycol molecule may function as the core.

The branching component may have a 4-armed configuration. For example, the core may have 4 arms and have a structure of:

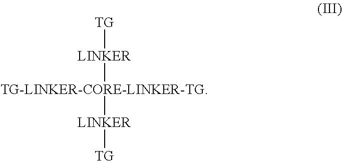

The branching component may have an 8-armed configuration. For example, the core may have 8 arms and have a structure of:

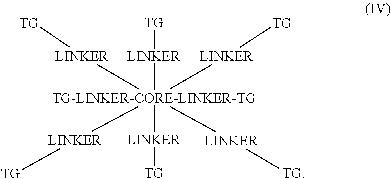

While the cores disclosed herein allow for three, four, five, six, seven, or eight arms (e.g., linkers), the number of arms can be further increased through additional modifications to the core. For example, PEG molecules can be pre-reacted to provide branched structures that each provide an available oxygen for each arm to attach.

In some examples, the branching component may include a multi-arm (e.g., 2, 4, or 8-arm) core having PEG linkers, and having tethering groups on one or more of the linkers. The one or more tethering groups may include any of the tethering groups one or more of NHS, NHS-SCM, NHS-SS, or NHS-SG. In specific examples, the branching component includes an 8-arm PEG having tethering groups including one or more of NHS, NHS-SCM, NHS NHS-SS, NHS-SG, or an ester of any of the foregoing including a portion of the linker (e.g., PEG-NHS ester).

The at least one branching component may have a molecular weight of at least about 1,000 Da (g/mol), such as about 1,000 Da to about 50,000 g/mol, about 1,000 Da to about 10,000 g/mol, about 10,000 Da to about 30,000 Da, 15,000 Da to about 25,000 Da, about 20,000 Da to about 40,000 Da, about 30,000 Da to about 50,000 Da, less than about 50,000 Da, or less than 30,000 Da.

In some examples, the branching component and resulting hydrogel may include a tethering group of a succinimidyl glutarate, a PEG linker, and a glycerol or glycerol derived core. The succinimidyl glutarate can be an NHS-SG. The branching component can include 2, 4, or 8 arms, such as NHS-PEG-SG-2, NHS-PEG-SG-4, and NHS-PEG-SG-8, respectively.

In some examples, the branching component and resulting hydrogel may include a tethering group of a succinimidyl carboxymethyl, a PEG linker, and a glycerol or glycerol derived core. The succinimidyl carboxymethyl can be an NHS-SCM. The branching component can include 2, 4, or 8 arms or NHS-PEG-SCM-2, NHS-PEG-SCM-4, and NHS-PEG-SCM-8, respectively.

In some examples, the branching component and resulting hydrogel may include a tethering group of a succinimidyl succinate (SS), a PEG linker, and a glycerol or glycerol derived core. The succinimidyl succinate can be an NHS-SS. The branching component can include 2, 4, or 8 arms or NHS-PEG-SS-2, NHS-PEG-SS-4, and NHS-PEG-SS-8, respectively.

The branching component is provided in an amount effective to result in a hydrogel with about 2 to about 50% weight/volume (w/v) of the branching component, such as about 2% to about 10% w/v, about 3% to about 8% w/v, about 5% to about 15% w/v, about 15% to about 25% w/v, about 10% to about 20% w/v, about 15% to about 35% w/v, about 20% to about 40% w/v, about 30% to about 50% w/v, less than 50% w/v, or less than 30% w/v, or less than 10% w/v.

The branching component may be provided in dry form and may be hydrated before or contemporaneous with deployment of the hydrogel embolic into the human body. The branching component may be disposed in an aqueous solution, such as water, saline, or the like. The water of the aqueous solution may be of sufficient quality or purity to prevent or limit unwanted contaminants when placed within a tissue of the human body. Such pure water may reduce the risk of generating or exaggerating an immune response in the human body. Such water may include one or more of filtered water, deionized water, distilled water, reverse osmosis water, or the like. Such water may be used in the basic solution used to hydrate the complexing component.

Upon mixing, the complexing component and branching component bond to each other (e.g., polymerize, cross-link) to form at least a portion of the hydrogel embolic. The tethering group may be bonded or otherwise attached to the at least one immunoregulator. In some examples, the at least one immunoregulator may be entangled, carried, or otherwise physically bound in the hydrogel matrix (e.g., cross-linked polymer chains of complexing components and branching components) of the hydrogel embolic.

The hydrogel embolic may include any commercial hydrogel embolic, such as Obsidio Conformable Embolic (available from Boston Scientific of Marlborough Massachusetts, USA).

### Immunomodulators

At least one immunomodulator is carried by the hydrogel embolic, such as being bonded, entangled, graphed, encapsulated, or otherwise disposed thereon or therein. The at least one immunomodulator includes a material configured to have an effect on the immune system of a human body. Such effects may include enhancing anti-cancer properties, suppressing pro-cancer properties, or the like. The immunomodulators disclosed herein may act directly on cancer cells or may act by enhancing host immune system responses against cancers. Interestingly, one aspect of butyrate's mechanism of action appears to be upregulation of LL-37 to enhance immune activity. Additionally, other small molecules with anti-cancer properties such as some interleukins may be useful as systemic or targeted immunotherapies with or without chemical modification. However, many small molecules investigated for their anti-cancer properties have not been pursued for clinical use due to insufficient concentrations in target tissues due to systemic administration or unacceptably severe side effects.

The at least one immunomodulator may include butyrate, hydroxybutyrate, at least one cecropin, at least one interleukin selected form a subset of interleukins, one or more antibodies, or combinations of any of the foregoing as disclosed in more detail below. The at least one immunomodulator may be in polymer or small molecule form.

The at least one immunomodulator may include one or more of butyrate or hydroxybutyrate. The at least one of butyrate or hydroxybutyrate may include a polymerized form of butyrate or hydroxybutyrate. The polymerized form may be present as nanoparticles, microparticles, or small particles. The nanoparticles, microparticles, or small particles may include nanobeads, microbeads, or small beads. The at least one of butyrate or hydroxybutyrate may include an oligomer or co-polymer of butyrate or hydroxybutyrate. The at least one of butyrate or hydroxybutyrate may include a small molecule of one or more of butyrate or hydroxybutyrate.

The hydroxybutyrate may includes one or more of poly(4-hydroxybutyrate) (P4HB), (poly(3-hydroxybutyrate) (P3HB), or (poly(2-hydroxybutyrate) (P2HB). The hydroxybutyrate or butyrate may be present as a salt or acid of the hydroxy butyrate or butyrate. For example, the butyrate may include sodium butyrate. The hydroxybutyrate may include one or more of sodium 4-hydroxybutyrate, 4-hydroxybutyric acid, sodium 3-hydroxybutyrate, 3-hydroxybutyric acid, sodium 2-hydroxybutyrate, or 2-hydroxybutyric acid.

The one or more of butyrate or hydroxybutyrate may be present in the hydrogel embolic at a concentration of at least about 0.05 millimolar (mM) of the embolic composition, such as about 0.05-48.0 mM, about 0.1-48.0 mM, about 12.0-48.0 mM, about 0.05-24.0 mM, about 0.25-24.0 mM, about 12.0-24.0 mM, about 0.05-24.0 mM, about 0.5-12.0 mM, about 2.0-12.0 mM, about 0.05-4.0 mM, about 0.05-0.5 mM, about 0.05-0.1 mM, 0.1-48.0 mM, less than about 48.0 mM, less than about 20.0 mM, or less than about 4.0 mM of the embolic composition. More than one of the butyrate or hydroxybutyrate may be present in the embolic composition. In such examples, each of the butyrate or hydroxybutyrate may be present in the above concentrations.

In some examples, the concentrations of the immunomodulators listed herein may be the minimum target concentration of the immunomodulator(s) in the tissue to be treated. In some examples, the concentration(s) of immunomodulator(s) in the embolic composition may be delivered to the tissue to be treated and only some of the immunomodulator(s) may be taken into the tissue to be treated. Accordingly, any of the concentrations for immunomodulator(s) disclosed herein may be present in the embolic composition in greater amounts or concentrations than those listed, such as at least 2 times more than the concentrations disclosed, such as at least 3 times, at least 5 times, or at least 10 times more than the concentrations listed. Such examples may provide sufficient concentration of immunomodulator(s) in the embolic composition to achieve a selected concentration (e.g., minimum concentration) of immunomodulator(s) in the tissue to be treated.

In some examples, the butyrate may be present in a concentration of about 0.05-4 mM, about 0.05-0.5 mM, or about 0.05-0.1 mM of the embolic composition. The P4HB may be present in a concentration of about 0.05-48.0 mM, about 0.1-48.0 mM, or about 12.0-48.0 mM of the embolic composition. The P3HB may be present in a concentration of about 0.05-24.0 mM, about 0.25-24.0 mM, or about 12.0-24.0 mM of the embolic composition. The P2HB may be present in a concentration of about 0.05-24.0 mM, about 0.5-12.0 mM, or about 2.0-12.0 mM of the embolic composition.

The concentrations of immunomodulators disclosed herein includes the amount of immunomodulator in the embolic composition or may be the concentration specific to local delivery and may be the target concentrations delivered to a target site within the human body. For example, these (local) concentrations may encompass any systemic delivery that results in the disclosed concentrations in the target tissue. In some examples, the concentrations of immunomodulators disclosed herein are listed as mass per unit of tumor mass.

In some examples, the oligomer or co-polymer of one or more of P4HB, P3HB, P2HB, or butyrate may include an oligomer or co-polymer of one or more of P4HB, P3HB, or P2HB in particle form and having a concentration of 0.05 mM to 2.0 mM of the embolic composition. In some examples, the oligomer or co-polymer of one or more of P4HB, P3HB, P2HB, or butyrate includes an oligomer or co-polymer of butyrate in particle form and having a concentration of 0.05 mM to 4.0 mM of the embolic composition. In some examples, the at least one of butyrate or hydroxybutyrate includes a polymerized form of butyrate having a concentration of 0.05 mM to 4.0 mM of the embolic composition and a polymerized form of hydroxybutyrate (e.g., P4HB, P3HB, or P2HB) having a concentration of 0.05 mM to 24.0 mM of the embolic composition.

The at least one immunomodulator may include one or more small molecules bonded, entrapped, grafted, or otherwise incorporated into the hydrogel embolic. For example, the hydrogel embolic may include a hydrogel backbone, such as any of the hydrogel backbones disclosed herein (e.g., complexing component(s) bonded to the branching component(s)) and the one or more small molecules used for the at least one immunomodulator may be bound to or otherwise attached to the hydrogel backbone via the tethering component. The one or more small molecules may be composed and attached to the hydrogel backbone in such a way as to release the small molecule(s) from the hydrogel backbone or tethering group thereof upon a chemical reaction. For example, the small molecule(s), such as butyrate or hydroxybutyrate, may be formulated to release form the hydrogel backbone via a chemical reaction. In such examples, the chemical reaction may include one or more of hydrolysis, enzymatic cleavage, or the like. Release of the small molecule from the hydrogel embolic allows for targeted treatment in selected tissues with the human body.

The at least one of butyrate or hydroxybutyrate may include one or more small molecules of at least one of butyrate or hydroxybutyrate. For example, the small molecule(s) may include hydroxybutyrate bonded to the tethering group and having a concentration of 0.05 mM to 24.0 mM of the embolic composition. The small molecule(s) may include butyrate having a concentration of 0.05 mM to 4.0 mM of the embolic composition.

In some examples, the small molecule(s) may be present in a concentration of at least about 0.01 micromolar (μM) of the embolic composition, such as about 0.01-2000.0 μM, about 0.05-40.0 μM, about 0.1-48.0 mM, about 100.0-2000.0 μM, about 200.0-1000.0 μMm, about 220.0-440.0 μM, about 1.0-50.0 μM, about 4.0-50.0 μM, about 25.0-50.0 μM, about 8.0-128.0 μM, about 8.0-64.0 μM, about 16.0-32.0 μM, about 0.01-160.0 μM, about 0.05-80.0 μM, about 0.05-40.0 μM, less than about 2000.0 μM, less than about 1000.0 μM, less than 440 μM, less than about 160 μM, less than 50.0 μM, or less than 20 μM of the embolic composition. More than one of the small molecules may be present in the embolic composition. In such examples, each of the small molecules may be present in the above concentrations.

The one or more small molecules may include at least one cecropin. The at least one cecropin may include one or more cathelicidins, one or more β-pleated sheet peptides, one or more random coil peptides, one or more cyclic peptides, or combinations of any of the foregoing. In some examples, the one or more cathelicidins include cathelicidin LL-37. In some examples, the one or more β-pleated sheet peptides includes a lactoferrin such as at least one of LfcinB, MPLfcinB6, LfcinB-P13, or the like. In some examples, the one or more random coil peptides include at least one alloferon such as one or more of KW-WK, PR-39, or PR-35. In some examples, the one or more cyclic peptides may include one or more diffusa cytides such as at least one of diffusa cytides 1-3, H-10, or RA-XII.

In some examples, the cathelicidins may be present in a concentration of about 100-2000 μM, about 200-1000 μM, or about 220-440 μM. In some examples, the β-pleated sheet peptides may be present in a concentration of about 1-50 μM, about 4-50 μM, or about 25-50 μM. In some examples, the random coil peptides may be present in a concentration of about 8-128 UM, about 8-64 μM, or about 16-32 μM. In some examples, the cyclic peptides may be present in a concentration of about 0.01-160 μM, about 0.05-80 μM, or about 0.05-40 μM.

In some examples, the one or more small molecules may include at least one interleukin, such as one or more of IL-2, IL-7, IL-10, IL-12, IL-15, IL-18, IL-21, IL-23, IL-24, IL-28, or IL-29. The one or more interleukins may be present in the embolic composition in a concentration of at least 5 picograms per gram (pg/g) of the embolic composition, or of tissue to be treated (e.g., tumor), such as about 5 pg/g to 3000 micrograms per gram (μg/g), about 100 μg/g to 3,000 μg/g, about 200 μg/g to 2,000 μg/g, about 300 μg/g to 1,000 μg/g, about 5 pg/g to 500 pg/g, about 10 pg/g to 1,000 pg/g, about 10 pg/g to 300 pg/g, about 50 pg/g to 100 pg/g, about 50 pg/g to 750 pg/g, about 250 pg/g to 300 pg/g, about 0.5 nanograms per gram (ng/g) to 2,000 ng/g, about 0.5 ng/g to 200 ng/g, about 2 ng/g to 50 ng/g, about 5 ng/g to 20 ng/g, about 1 ng/g to 30 ng/g, about 1 ng/g to 10 ng/g, about 1 ng/g to 3 ng/g, about 10 ng/g to about 2000 ng/g, about 5 ng/g to 1,000 ng/g, about 100 ng/g to 500 ng/g, about 40 ng/g to 400 ng/g, about 40 ng/g to 160 ng/g, about 40 ng/g to 80 ng/g, about 0.5 ng/g to 200 ng/g, about 2 ng/g to 500 ng/g, about 5 ng/g to 20 ng/g, less than about 3,000 μg/g, less than about 2000 ng/g, less than about 1,000 pg/g, less than 500 pg/g. As noted above, any of the immunomodulators disclosed herein may be present in the embolic composition in a greater concentration or amount than is listed, such as at least two times greater (e.g., at least 5 times or at least 10 times) than those amounts listed. Such examples may be particularly useful in reaching a target concentration in the tissue to be treated.

In some examples, the at least one interleukin includes IL-2 in a concentration of at least about 10 pg/g, about 100 μg/g to 3,000 μg/g, about 200 μg/g to 2,000 μg/g, or about 300 μg/g to 1,000 μg/g. In some examples, the at least one interleukin includes IL-4 in a concentration of about 1 ng/g to 30 ng/g, about 1 ng/g to 10 ng/g, or about 1 ng/g to 3 ng/g. In some examples, the at least one interleukin includes IL-7 in a concentration of at least about 1 pg/g or about 45 pg/g to 90 pg/g. In some examples, the at least one interleukin includes IL-10 in a concentration of about 5 pg/g to 500 pg/g, about 10 pg/g to 300 pg/g, or about 50 pg/g to 100 pg/g. In some examples, the at least one interleukin includes IL-12 in a concentration of at least about 500 pg/g, about 10 pg/g to 1,000 pg/g, about 50 pg/g to 750 pg/g, or about 250 pg/g to 300 pg/g. In some examples, the at least one interleukin includes IL-15 in a concentration of about 0.5 ng/g to 200 ng/g, about 2 ng/g to 50 ng/g, or about 5 ng/g to 20 ng/g. In some examples, the at least one interleukin includes IL-18 in a concentration of about 0.5 ng/g to 200 ng/g, about 2 ng/g to 50 ng/g, or about 5 ng/g to 20 ng/g. In some examples, the at least one interleukin includes IL-21 in a concentration of about 10 ng/g to about 2000 ng/g, about 5 ng/g to 1,000 ng/g, or about 100 ng/g to 500 ng/g. In some examples, the at least one interleukin includes IL-23 in a concentration of at least about 40 ng/g, about 40 ng/g to 400 ng/g, about 40 ng/g to 160 ng/g, or about 40 ng/g to 80 ng/g. In some examples, the at least one interleukin includes IL-27 in a concentration of about 0.5 ng/g to 200 ng/g, about 2 ng/g to 50 ng/g, about 5 ng/g to 20 ng/g.

In some examples, the immunoregulator includes one or more antibodies configured to block pro-cancer cell receptors. The one or more antibodies may include at least one of anti-VEGF, anti-IL-1RA, anti-IL-4, anti-IL-6, or anti-IL-7. The one or more antibodies may be present in the embolic composition at a concentration of at least about 1 pg/g, such as about 1 pg/g to 4 μg/g, about 0.05 to 4 μg/g, about 0.1-1.0 μg/g, about 0.25-0.5 μg/g, about 1-500 pg/g, about 5-200 ng/g, about 20-150 ng/g, about 120-1200 pg/g, about 240-600 pg/g, about 360-480 pg/g, about 10-1,000 ng/g, about 100-800 ng/g, about 400-600 ng/g, at least about 0.05 μg/g, at least about 10 ng/g, at least about 120 pg/g, less than 4 μg/g, less than 1000 ng/g, less than 600 pg/g, or less than 250 pg/g.

In some examples, the one or more antibodies include at least one of anti-VEGF at a concentration of at least about 0.05 μg/g of the hydrogel embolic, anti-IL-1RA at a concentration of at least about 10 ng/g of the hydrogel embolic, anti-IL-4 at a concentration of at least about 1 ng/g, anti-IL-6 at a concentration of at least about 120 pg/g of the hydrogel embolic, or anti-IL-7 at a concentration of at least about 1 pg/g of the hydrogel embolic. In some examples, the one or more antibodies may include anti-VEGF at a concentration of about 0.05-4 μg/g, about 0.1-1.0 μg/g, or about 0.25 to 0.5 μg/g. In some examples, the one or more antibodies may include anti-1L-1RA at a concentration of 10-1,000 ng/g, 100-800 ng/g, or 400-600 ng/g. In some examples, the one or more antibodies may include anti-1L-6 at a concentration of about 120-1200 pg/g, about 240-600 pg/g, or about 360-480 pg/g. In some examples, the one or more antibodies may include anti-1L-7 at a concentration of about 1-500 pg/g, about 5-200 ng/g, or about 20-150 ng/g.

Any and all possible combinations of the one or more antibodies may be utilized and concentrations of the individual anti-bodies would not necessarily change when used in combination.

Any combination of immunomodulators disclosed herein in any of the concentrations disclosed herein may be present in the embolic composition. For example, greater efficacy of cancer treatments may be achieved by utilizing more than one of any immunomodulators (e.g., small molecules) disclosed herein. Any combination of immunomodulators disclosed herein is anticipated to induce an additive or greater than additive effect based on complimentary immune stimulation through multiple immunomodulatory/anti-cancer signaling pathways.

As referenced above, the degredation-mediated release of immunomodulators bound to the embolic hydrogel or other materials disclosed herein (e.g., butyrate/hydroxybutyrate and their oligomers-co-polymers) may be via one or more of hydrolysis, enzymatic cleavage, or surface erosion of polymer units followed by diffusion through the hydrogel and then surrounding tissues. Accordingly, local delivery in a target tissue (e.g., tumor) may be achieved. Any of the small molecules above such as cecropins, interleukins, and antibodies against interleukin receptors may be modified to increase half-life, control binding to embolic materials such as resins or hydrogels, and/or alter in vivo activity (e.g. increase binding affinity to target). These modifications may include PEGylation (polyethylene glycol), XTENylation, PTTGylation (poly(thioglycidyl glycerol), polysarcosine bioconjugation, micelle encapsulation, enclosure/entrapment in micropores, etc. The Small molecules disclosed herein or their modified forms (e.g., chemically bonded forms) may be chemically incorporated into the embolic hydrogel and released via chemical linkages such as succinimides, carbodiimides, maleimides, propionamides, pyridyl disulfides, α-haloacetyls, etc. Such chemical linkages may include one or more of covalent bonds, Van der Waals force, hydrogen bonds, ionic bonds, or the like.

In some embodiments, the embolic composition includes embolic beads, such as small beads, microbeads, or nanobeads having at least one immunomodulator thereon or therein. Similarly, to the hydrogel-based examples disclosed herein having the immunomodulators disclosed herein may be additionally or alternatively incorporated onto or into microbeads, nanobeads, or small beads sized, shaped, and composed for embolic delivery to a selected tissue in the human body. The average diameter of the microbeads may be on a micron scale such as at least 1 μm, 1 μm-500 μm, 10 μm-100 μm, or less than 500 μm. The average diameter of the small beads may be larger than the micron scale, such as having an average diameter of at least 1 mm, 1 mm-10 mm, 1 mm-5 mm, 5 mm-10 mm, or less than 500 μm. In some examples, the average diameter of the beads may be on the nanoscale.

The embolic beads may include a polymer of any of the hydrogels or other polymerizable materials disclosed herein. Embolic microbeads may be formed from or coated with a polymerized form of any of the above immunomodulators, such as hydroxybutyrate or butyrate, cecropins, or interleukins. For example, microbeads suitable for embolic delivery may include a polymerized butyrate or hydroxybutyrate in the shape of beads on a micron scale. In some examples, the embolic beads may include hydrogel beads composed of any of the hydrogel embolics disclosed herein. One or more of any of the immunomodulator(s) disclosed herein may be disposed on or in the embolic beads, such as via any of the chemical bonds, entanglements, physical bonds, or other means of retention disclosed herein.

The embolic beads may be delivered to a target tissue alone or with any of the hydrogel embolics disclosed herein.

The efficacy of the examples disclosed herein for treatment of cancer is dependent upon the cancer type and specific humoral expression of the tumor cells targeted. Concentrations of the immunomodulators may be selected based on the cancer type and humoral expression of the tumor cells. Concentrations of the immunomodulators disclosed herein may be the same regardless of state, such as oligomers, co-polymers, other polymers, or as small molecules. The inventors currently believe the immunomodulators disclosed herein will have anti-cancer and immunomodulating effect when in the monomer, oligomer (short chain), or small molecule state.

Embolic agents or compositions such as resin beads or hydrogels may be modified to incorporate any of the immunomodulators disclosed herein, either individually or in combination, such as by chemical grafting or tethering, embedding, or encapsulation. The embolic agent may then be administered using a traditional embolization technique, such as transarterial embolization, to place the embolic agent and incorporated immunomodulator(s) within the vasculature local to the target tumor or tissue. The presence of the immunomodulator(s) is expected to locally elicit an elevated immune response to the tumor and cancer cells, resulting in accelerated tumor shrinkage, earlier remission, increased patient longevity, and/or enhanced activity of other anti-cancer/anti-carcinogenic treatments.

The embodiments disclosed herein include treatment of tumors via embolic agents comprising one or more of the immunomodulators disclosed herein.

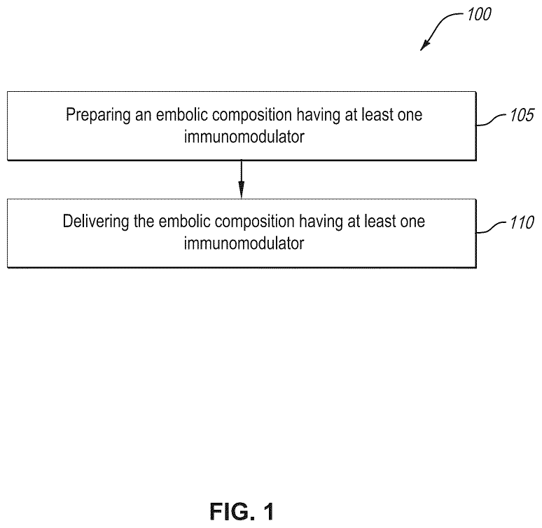

FIG. 1 is a flow chart of a method 100 of treating a tumor in a human body, according to an embodiment. The method 100 includes an act 105 of preparing an embolic composition having at least one immunomodulator thereon at least locally within the human body via an embolization; and an act 110 of delivering the embolic composition having at least one immunomodulator thereon at least locally within the human body via an embolization. In some examples, the method 100 may include more or fewer acts than acts 105 and 110. For example, the act 105 may be omitted or the acts 105 or 110 may be split into multiple acts.

The act 110 of delivering an embolic composition having at least one immunomodulator thereon at least locally within the human body via an embolization may include using any of the embolic compositions disclosed herein. For example, the embolic composition may include a hydrogel embolic and at least one immunomodulator disposed within the hydrogel embolic. The hydrogel embolic may include any of the hydrogel embolics or beads disclosed herein.

The at least one immune modulator may include any of the at least one immunomodulators disclosed herein in any of the concentrations disclosed herein. For example, the at least one immunomodulator may include one or more of butyrate or hydroxybutyrate in a concentration of 0.05 millimolar (mM) to 48.0 mM of the embolic composition.

In some examples, the hydrogel embolic includes a hydrogel backbone and the at least one immunomodulator includes a small molecule form of the at least one of butyrate or hydroxybutyrate attached to the hydrogel backbone via a tethering group composed to release the small molecule form of the at least one of butyrate or hydroxybutyrate via a chemical reaction. The at least one immunomodulator may include one or more small molecules having immunomodulatory or anti-cancer properties. In such examples, the one or more small molecules may include at least one of butyrate, hydroxybutyrate, one or more cecropins, one or more interleukins with anti-cancer properties, or one or more antibodies capable of blocking pro-cancer interleukins or their corresponding cell receptors.

The embolization may include one or more of transarterial embolization, conventional transarterial chemoembolization, transarterial radioembolization, transarterial radioembolization, or drug-eluting bead transarterial chemoembolization. In some examples, delivering an embolic composition having at least one immunomodulator thereon at least locally within the human body via an embolization includes injecting the one or more hydrogel compositions into a selected region or tissue (e.g., tumor) of the human body. The embolization may include injecting the embolic composition into the selected tissue via a catheter, needle, γ-connector, or the like

In some examples, delivering an embolic composition having at least one immunomodulator thereon at least locally within the human body via an embolization includes systemically delivering the one or more hydrogel compositions into the human body. Such systemic delivery may include a non-targeted treatment such as into a vein, artery, organ, or muscle.

The act 105 of preparing the embolic composition may include mixing components of the one or more hydrogel embolics. Preparing the embolic composition may include forming any of the hydrogel embolics disclosed herein. Preparing the embolic composition may include adding the immunomodulator(s) to the hydrogel embolic(s). Preparing the one or more hydrogel embolics may include hydrating one or more of the complexing component (e.g., albumin) or polyethylene glycol. Such hydration may include one or more of adding a basic solution to the complexing component and adding an aqueous solution to the branching component of the hydrogel embolic.

The hydrogel embolic may include a hydrogel mixture or hydrogel beads as disclosed herein.

While many examples are disclosed herein, a number of specific examples are disclosed below.

### PROPHETIC EXAMPLES

In an example, the hydrogel embolic may include any commercialized hydrogel embolic into which poly(4-hydroxybutyrate) (P4HB) microbeads of 10-100 μm diameter are dispersed prior to deployment or are deployed into the vasculature simultaneously with the hydrogel and entrapped therein during in situ crosslinking. For example, a 1 ml Obsidio Conformable Embolic may be interspersed with 100 μg P4HB microbeads of 10 μm diameter via mixing prior to embolic composition (hydrogel) deployment or via simultaneous introduction through a Y-connector causing thorough intermixing during deployment.

In an example, a small molecule form of hydroxybutyrate or butyrate is engrafted onto the hydrogel backbone of the embolic composition via tethering groups which release the small molecule upon degradation by chemical cleavage. The hydrogel backbone includes at least one of albumin and a multi-arm PEG (e.g., 4-arm or 8-arm). The hydroxybutyrate or butyrate may be about 5-40 percent by weight of the embolic composition and may be of the form poly(4-hydroxybutyrate). In such examples, the hydrogel backbone and tethering linkages may comprise about 60-95 percent by weight of the embolic composition, and the tethering linkages include one or more succinimides (e.g., N-hydroxy succinimide conjugated with PEG (e.g., NHS ester PEG), maleimides (e.g., maleimide PEG), or propionamides (e.g., propionamide PEG), to release the hydroxybutyrate or butyrate upon degradation by chemical cleavage via one or more of hydrolysis or enzymatic degradation. For example, this example includes 5.0 ml of rHSA solution at pH about 9.5 (750 mg rHSA in 5.0 ml water) homogeneously mixed with 500 mg of 4-hydroxybutyrate, and then combined with 525 mg of 8-arm PEG succinimidyl glutarate ester at each terminal, 20K MW) in 5 ml water, yielding an embolic hydrogel randomly engrafted with 4-hydroxybutyrate which releases upon hydrolysis of the embolic hydrogel.

In an example, a small molecule with immunomodulatory properties such as cathelicidin LL-37, lactoferrin, or alloferon is engrafted onto the hydrogel backbone with tethering groups composed to release the small molecule upon degradation by chemical cleavage. For example, a 525 mg amount of 8-arm PEG (PEG-SG-8, may have succinimidyl glutarate ester at each terminal, 20K MW), is homogeneously mixed with 50 mg of PEGylated cathelicidin LL-37, add 5.0 ml water, and then combined with 5.0 ml of rHSA solution at pH about 9.5 (750 mg rHSA in 5 ml water), yielding an embolic hydrogel randomly engrafted with cathelicidin LL-37 which released from the embolic hydrogel release upon hydrolysis. As an alternative example, the cathelicidin LL-37 could be biotinylated.

In an example, the hydrogel comprises any commercialized hydrogel embolic into which a solution of IL-4 and IL-12 is dispersed prior to deployment or is deployed into the vasculature simultaneously with the hydrogel embolic and entrapped therein during in situ crosslinking. For example, 1 ml Obsidio Conformable Embolic may be homogeneously mixed with 20 μl normal saline containing 1 ng IL-4 and 50 μg IL-12 prior to hydrogel deployment or via simultaneous introduction through a Y connector causing thorough intermixing during deployment.

In an example, a small antibody capable of blocking pro-cancer interleukins such as anti-VEGF is entrapped within a hydrogel embolic and released via diffusion. A 5.0 ml quantity of rHSA solution at pH of about 9.5 (750 mg rHSA in 5.0 ml water) are homogeneously mixed with 40 μg anti-VEGF, and then combined with 525 mg of 8-arm PEG succinimidyl glutarate ester at each terminal, 20K MW) in 5 ml water, yielding an embolic hydrogel from which anti-VEGF releases via diffusion.

In an example, the embolic composition comprises any commercialized resin microbead embolic onto which a coating of P4HB is deposited prior to deployment into the vasculature. The microbead may include PEG or gelatin microbeads and, via spray coating of melted or dissolved P4HB, deposited in a 2.0 μm layer prior to microbead deployment through a standard catheter.

By administering the embolic compositions disclosed herein locally, issues with systemic administration including severe side effects and insufficient concentrations in the target tissues/tumor may be avoided. A local administration of the embolic compositions disclosed herein includes localized delivery of immunomodulator(s) bound to a hydrogel embolic acting as the carrier.

As used herein, the term “about” or “substantially” refers to an allowable variance of the term modified by “about” by ±10% or ±5%. Further, the terms “less than,” “or less,” “greater than”, “more than,” or “or more” include as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

What is claimed is:

1. An embolic composition, comprising:
a hydrogel embolic; and
at least one immunomodulator disposed within the hydrogel embolic.

2. The embolic composition of claim 1 wherein the at least one immunomodulator includes one or more of butyrate or hydroxybutyrate in a concentration of 0.05 millimolar (mM) to 48.0 mM of the embolic composition.

3. The embolic composition of claim 2 wherein the one or more of butyrate or hydroxybutyrate includes a polymerized form of butyrate as nanoparticles, microparticles, or small molecules at a concentration of 0.05 mM to 4.0 mM of the embolic composition.

4. The embolic composition of claim 2 wherein the one or more of butyrate or hydroxybutyrate includes a polymerized form of hydroxybutyrate as nanoparticles, microparticles, or small particles.

5. The embolic composition of claim 4 wherein the hydroxybutyrate includes one or more of poly(4-hydroxybutyrate) (P4HB), (poly(3-hydroxybutyrate) (P3HB), or (poly(2-hydroxybutyrate) (P2HB).

6. The embolic composition of claim 2 wherein the one or more of butyrate or hydroxybutyrate includes an oligomer or co-polymer of one or more of P4HB, P3HB, P2HB, or butyrate.

7. The embolic composition of claim 6 wherein the oligomer or co-polymer of one or more of P4HB, P3HB, P2HB, or butyrate includes an oligomer or co-polymer of one or more of P4HB, P3HB, or P2HB in particle form and having a concentration of 0.05 mM to 2.0 mM of the embolic composition.

8. The embolic composition of claim 6 wherein the oligomer or co-polymer of one or more of P4HB, P3HB, P2HB, or butyrate includes an oligomer or co-polymer of butyrate in particle form and having a concentration of 0.05 mM to 4.0 mM of the embolic composition.

9. The embolic composition of claim 2 wherein the one or more of butyrate or hydroxybutyrate includes:
a polymerized form of butyrate having a concentration of 0.05 mM to 4.0 mM of the embolic composition; and
a polymerized form of hydroxybutyrate having a concentration of 0.05 mM to 24 mM of the embolic composition.

10. The embolic composition of claim 9 wherein the polymerized form of hydroxybutyrate includes an oligomer or co-polymer of one or more of P4HB, P3HB, or P2HB.

11. The embolic composition of claim 2 wherein the hydrogel embolic includes:
a hydrogel backbone; and
the at least one immunomodulator includes a small molecule form of the one or more of butyrate or hydroxybutyrate attached to the hydrogel backbone via a tethering group composed to release the small molecule form of the at least one of butyrate or hydroxybutyrate via a chemical reaction.

12. The embolic composition of claim 11 wherein the chemical reaction includes one or more of hydrolysis or enzymatic cleavage.

13. The embolic composition of claim 11 wherein the tethering group includes one or more of a succinimide, a carbodiimide, a maleimide, a propionamide, a pyridyl disulfide, or an α-haloacetyl.

14. The embolic composition of claim 11 wherein the small molecule form includes hydroxybutyrate having a concentration of 0.05 mM to 24.0 mM of the embolic composition.

15. The embolic composition of claim 11 wherein the small molecule form includes butyrate having a concentration of 0.05 mM to 4.0 mM of the embolic composition.

16. The embolic composition of claim 11 wherein the hydrogel backbone includes one or more of albumin or a polyethylene glycol.

17. The embolic composition of claim 16 wherein the albumin includes recombinant human serum albumin and the polyethylene glycol includes a multi-arm polyethylene glycol.

18. The embolic composition of claim 16 wherein the albumin includes recombinant human serum albumin and the polyethylene glycol includes an 8-arm polyethylene glycol having an average molecular weight of at least about 20,000 Da.

19. The embolic composition of claim 1 wherein the at least one immunomodulator includes one or more small molecules having immunomodulatory or anti-cancer properties.

20. The embolic composition of claim 19 wherein the one or more small molecules are entrapped, grafted, or incorporated into the hydrogel embolic.

21. The embolic composition of claim 19 wherein the hydrogel embolic includes one or more of albumin or polyethylene glycol.

22. The embolic composition of claim 19 wherein the one or more small molecules include at least one cecropin.

23. The embolic composition of claim 22 wherein the at least one cecropin includes one or more of a cathelicidin, a β-pleated sheet peptide, a random coil peptide, or a cyclic peptide.

24. The embolic composition of claim 22 wherein the one or more small molecules include at least one of cathelicidin LL-37, lactoferrin, alloferon, or diffusa cytides, in a concentration of 0.05 μM to 40 μM.

25. The embolic composition of claim 19 wherein the one or more small molecules includes at least one of a subset of interleukins including IL-2, IL-7, IL-10, IL-12, IL-15, IL-18, IL-21, IL-23, IL-24, IL-28, or IL-29.

26. The embolic composition of claim 25 wherein the at least one of a subset of interleukins includes one or more of IL-2 at a concentration of at least 10 picograms per gram (pg/g) of the embolic composition, IL-7 at a concentration of 45 to 90 pg/g of the embolic composition, IL-12 at a concentration of at least 500 pg/g of the embolic composition, or IL-23 at a concentration of at least 40 nanograms per gram (ng/g) of the embolic composition.

27. The embolic composition of claim 1 wherein the immunoregulator includes one or more antibodies configured to block pro-cancer cell receptors.

28. The embolic composition of claim 27 wherein the one or more antibodies include at least one of anti-VEGF at a concentration of at least about 0.05 μg/g of the hydrogel embolic, anti-IL-1RA at a concentration of at least about 10 ng/g of the hydrogel embolic, anti-IL-4 at a concentration of at least about 1 ng/g, anti-IL-6 at a concentration of at least about 120 pg/g of the hydrogel embolic, or anti-IL-7 at a concentration of at least about 1 pg/g of the hydrogel embolic.

29. Embolic microbeads comprising a polymerized form of the embolic composition of claim 1.

30. A method of treating a tumor in a human body, the method comprising:
delivering an embolic composition having at least one immunomodulator thereon at least locally within the human body via an embolization, the embolic composition including:
a hydrogel embolic; and
at least one immunomodulator disposed within the hydrogel embolic.

31. The method of claim 30 wherein the at least one immunomodulator includes one or more of butyrate or hydroxybutyrate in a concentration of 0.05 millimolar (mM) to 48.0 mM of the embolic composition.

32. The method of claim 30 wherein the hydrogel embolic includes:
a hydrogel backbone; and
the at least one immunomodulator includes a small molecule form of at least one of butyrate or hydroxybutyrate attached to the hydrogel backbone via a tethering group composed to release the small molecule form of the at least one of butyrate or hydroxybutyrate via a chemical reaction.

33. The method of claim 32 wherein the at least one immunomodulator includes one or more small molecules having immunomodulatory or anti-cancer properties.

34. The method of claim 33 wherein the one or more small molecules include at least one of butyrate, hydroxybutyrate, one or more cecropins, one or more interleukins with anti-cancer properties, or one or more antibodies capable of blocking pro-cancer interleukins or their corresponding cell receptors.

35. The method of claim 30 wherein the embolization includes one or more of transarterial embolization, conventional transarterial chemoembolization, transarterial radioembolization, transarterial radioembolization, or drug-eluting bead transarterial chemoembolization.

36. The method of claim 30 wherein delivering an embolic composition having at least one immunomodulator thereon at least locally within the human body via an embolization includes injecting the one or more embolic compositions into a selected region of the human body.

37. The method of claim 30 wherein delivering an embolic composition having at least one immunomodulator thereon at least locally within the human body via an embolization includes systemically delivering the one or more embolic compositions into the human body.

38. The method of claim 30 further comprising preparing the embolic composition.

39. The method of claim 34 wherein the embolic composition includes mixing components of the one or more hydrogel embolic.

40. The method of claim 34 wherein preparing the one or more embolic compositions includes hydrating one or more of albumin or polyethylene glycol.
